(19) United States
(12) Patent Application Publication
(10) Pub. No.: US 2012/0186411 A1
(43) Pub. Date: Jul. 26, 2012

(54) AUTOMATED SURGICAL ROD CUTTER AND BENDER INCLUDING A POWER-BASE, ASSEMBLY FOR ROD CUTTING, AND ASSEMBLY FOR ROD BENDING

(76) Inventors: Sebstian Lodahi, (US); Ryan Leard, (US); Chris Karas, (US)

(21) Appl. No.: 13/355,737

(22) Filed: Jan. 23, 2012

Related U.S. Application Data
(60) Provisional application No. 61/461,942, filed on Jan. 25, 2011.

(51) Int. Cl. A61B 17/56; B21D 7/00; B23D 23/00

(52) U.S. Cl. 83/452; 83/523; 83/639.1; 83/571; 72/453.01

(57) ABSTRACT

A rod manipulator including power-base and various assemblies, interchangeably attaching to the base. The power-base is composed of a pneumatic cylinder and piston which affects the function of various assemblies. The piston moves the central component of an assembly toward the fixed portion of that assembly. A bending assembly containing mobile pivots around which a surgical metal rod can be bent. A cutting assembly containing blades in central and fixed portions between which a surgical metal rod can be cut. The distal end of the cut rod is retained by replaceable, sterilizable, eject-grips during and immediately after the cutting operation. The general object of the invention is to provide an improved surgical rod cutter and surgical rod bender, capable of performing both tasks with one power source, requires one-person operation, eliminates the need for significant muscular forces and eliminates the opportunity for an unsafe projectile in the operating room.

This document is the nonprovisional patent application for the following provision patent application:
Provisional Patent Application No. 61/461,942
Provisional Patent Application Date: Jan. 25, 2011

### FIELD OF THE INVENTION

This invention relates generally to a surgical rod cutter and surgical rod bender used to reduce the length and/or change the linearity of a surgically implanted metal rod. This specific invention is generally related to a rod cutting tool with one or two blades removably attached to a pneumatic cylinder and a rod bending tool with two positionable pivot points and one central thrust anvil removably attached to a pneumatic cylinder.

### BACKGROUND OF THE INVENTION

### Prior Art

A surgical rod cutter and surgical rod bender reduces the length or changes the linearity of surgical rod. High strength surgical rods are composed of a variety of materials including stainless steel, titanium, and cobalt-chrome alloys. High strength surgical rods are available from multiple manufacturers varying in length, cross-section, and linearity. While these manufactured rods are appropriate for a number of surgical operations, a large number of operations require custom rod manipulation to occur intra-operatively to fit a specific skeletal surgical construct. In spine surgery specifically, rods are used to connect and/or immobilize vertebrae for the purpose of spinal stabilization.

Currently the art of non-surgical rod manipulation by cutting or bending includes hand-held or table-mounted devices both manually actuated or automated. U.S. Pat. No. 4,369,576 describes a hydraulic, automated rod cutter for industrial purposes; which, is not appropriate for an operating room. This device is large and heavy, thus occupying much floor or counter space and would be difficult for operating room personnel to handle. Hydraulic pressurized power sources are not widely available in operating room. Furthermore, this device is not designed to be sterilized nor be used in the sterile field.

For the specific case of manipulating sterile surgical rods in the sterile operating field for the purpose of implantation into the human body the current art includes both table-mounted and hand-held devices. Said devices are often manually actuated, therefore requiring significant force by the operator to affect cutting or bending of the rod. Bolt cutters, in the form of compound levers and pivoted jaws have been the mainstay for cutting rods in the surgical environment. These devices do accomplish their intended operation, but not without disadvantages. Manual bolt cutters often require two operators in practice. One operator applies significant muscular force to the levers, increasing risk of injury or failure of operation. The muscular force required to operate some manual rod cutters and rod benders is great enough to limit the persons able to properly handle the devices. The second operator uses both hands to hold the rod ends to ensure rod alignment and stability while cutting. The second operator also attempts to prevent the cut segments from becoming projectiles. For the purpose of cutting short segments the operator's hands are dangerously close to the cutting blades and cannot contain the cut rod segments.

U.S. Pat. No. 5,261,303 is a table-mounted, manual rod cutter, wherein the rod is cut by a combination of offset shearing action and rotary wringing action. An advantage of this system is that it provides a mechanism for containing cut rod segments. Additionally the single-lever action allows for a distribution of force by the user which is less strenuous than operating a dual lever rod cutter. While this device reduces the force required to cut a rod over the dual lever devices, the force required is approximately 70 pounds of force. The continued requirement for significant muscular force by the user is a disadvantage. Also, a two-user operation is necessary given the distance of the lever end from the blades. Another disadvantage of this device is the lack of visual confirmation by line of sight to the cutting point immediately prior to and during the operation of cutting due to the rod being enclosed in an opaque housing. Similar to other prior art the blades are reused and integral to the device but are also prone to dulling or failing over time, therefore requiring periodic sharpening or entire device replacement.

Two-lever and one-lever manual options exist in various forms shown in U.S. Pat. Nos. 2,494,996, 3,333,338, 3,370,353, and 2,560,318. These devices share similar pivoting cutting action around a center pin as in U.S. Pat. No. 5,261,303. U.S. Pat. Nos. 2,543,018, 2,249,515, and 4,722,257 describe various hand-held and table mounted shearing devices. The aforementioned manual devices require two-user operation and considerable muscular force by one user regardless of whether the device is handheld, table-mounted, single-levered or double-levered. The high forces required are a function of the strength of the materials required for skeletal reconstruction.

None of the current surgical rod cutter devices accommodates a rod bending assembly.

Among current designs for rod bending are hand-held and table-mounted devices. A common hand-held, two-lever design is shown in U.S. Pat. No. 4,474,046. This tool uses one central anvil, around which two lateral pivots operated by the levers create a change in linearity in the rod. Given the necessarily rigid rod materials this requires significant force by the user and often requires two-user operation in which one user holds the rod and a second user utilizes both hands to apply force to the levers. A variation of this design is shown in U.S. Pat. No. 5,490,409 in which the central anvil around which the rod is bent is circumferentially adjustable allowing for limited varying arc angles. The significant muscular forces and two-person operation requirements are unchanged in this design.

A table-mounted model with multiple adjustable rod bending threaded bolts is described in U.S. Pat. No. 6,035,691. This tool allows for one-person use and decreases the muscular forces necessary through a single-lever design. Another advantage is the ability to manipulate the threaded bolts precisely to determine the extent of arc creation in the rod. The plurality of adjustable threaded bolts necessitates significant time commitment in preparing the proper arc for each rod bend. U.S. Pat. No. 6,644,087 describes a table-mounted, manual rod bender which creates an arc in the rod by forcing a single, mobile central piston against the rod portion to be bent, while stabilizing the lateral portions of the rod with adjustable pivots to create a range of arc depths. Given the rigidity of the rods available for surgical constructs, these methods require significant muscular forces by the user.

Another form of rod bender is shown in U.S. Pat. Nos. 5,161,404, and 5,389,099 and U.S. patent application Ser. No. 11/280,013. These surgical rod bending designs include tools with two separate, unconnected components which individually attach to the rod at two points between which a linearity change may be created in the rod by distracting or compacting the space between the two distal levers. The advantage of such systems is the ability to engage and bend the rod after implantation in the human body (in situ). Disadvantages include two-person operation when ex-situ and the need for significant muscular forces by the user.

One proposed mechanism for alleviating the muscular forces required by manual rod bending is to automate the process by which various components may create a change in linearity in the rod. U.S. patent application Ser. No. 11/355,593 describes a system which includes sensors which predict the preferred final linearity of the rod and a tool which uses electric automation to form the rod into that preferred final linearity. Rod bending is accomplished by means of multiple servomotors creating predetermined arcs against vertical discs. Advantages of such a system include a method for matching the template for preferred rod linearity to the final rod and creating said surgical rod without the use of significant muscular forces. Several disadvantages are the significant increases in complexity over prior art with respect to operation, sterilization, and cost. Furthermore this device does not perform rod cutting.

None of the current surgical rod bending devices accommodate a rod cutting assembly.

In order to address the disadvantages of prior art and achieve other objects in accordance with the purpose of the present invention as described herein, the automated surgical rod cutter and bender may comprise the following.

### DISCLOSURE OF THE INVENTION

This invention is directed to the art of surgical metal rod manipulation. Specifically, a rod-cutting assembly or rod-bending assembly is coupled with a power-base composed generally of housing and a pneumatic cylinder which transfers force via a piston to a cutting assembly or bending assembly ultimately affecting the rod length or linearity, respectively.

The general object of the invention is to provide an improved surgical rod cutter and surgical rod bender that is capable of performing both tasks with one power source, requires only one-person operation, eliminates the need for significant muscular forces and eliminates the opportunity for an unsafe projectile in the operating room.

Another object is that the rod manipulator's respective assemblies are of a size that said assemblies can be sterilized by conventional methods. The present embodiment does not require an increase in lever length to decrease the force required to affect the cut or bend. The plurality of assemblies obviates the need for multiple separate tools. The cutting assembly and bending assembly require sterilization while the base unit can be equally functional and accommodate both assemblies with a widely available, transparent sterile cover, eliminating the need for sterilization of the power-base and decreasing the significant wear sterilization may have on said device.

The pneumatically powered device allows for compatibility with widely available compressed air sources within the operating room that powers a plurality of current surgical devices.

Furthermore, the single-finger, push-button initiation and completion of bending or cutting allows for single-user operation with negligible muscular force. Each cut or bend is not affected by fatigue. The blades within the cutting assembly are removable and interchangeable between operations. Within prior art, the blades are integral to the device and not removable, predisposing such devices to significant wear over time, uses, and multiple sterilizations which will decrease the efficiency and ease of use of said device.

In addition, an important object with any surgical device is safety. Push-button operation allows the users more controlled, neutral, and ergonomic movements around dangerous equipment, sterile fields, and anesthetized patients. Eject-grips contain cut rod segments to eliminate the possibility of said segments becoming projectiles or entering a patient's open wound(s). Open assembly design allows full visualization of the predetermined segment of rod to be cut.

Furthermore, the open assembly, wedge-cut method of cutting the rod accommodates a plurality of rod widths, including all present widths of spinal rod instrumentation and other widths not heretofore available.

Another advantage of the present invention is the ability to move the lateral dynamic pivots on the bending assembly allowing for more precise changes in linearity of the rod.

### BRIEF DESCRIPTION OF THE DRAWINGS

In order to better appreciate how the advantages and object of the invention are obtained, a more particular description of the invention briefly described above will be rendered by reference to a specific embodiment thereof which is illustrated in the appended drawings. Understanding that these drawings depict only a typical embodiment of the invention at time of patent, and are not therefore to be considered limiting of its scope. The invention will be described and explained with additional specificity and detail through the use of the accompanying drawings in which:

FIG. 1 is a perspective view of the power-base of this invention without the cutting or bending assembly;

FIG. 2 is an overhead view of the power-base without the cover as to expose the internal components;

FIG. 3 is an overhead view of the cutting assembly;

FIG. 4 is a perspective view of the eject-grips relationship to the rod being cut as viewed from underneath the cutting assembly;

FIG. 5 is a cross section view of the blades relationship to the rod being cut;

FIG. 6 is a perspective exploded view of the main components of the bending assembly;

FIG. 7 is an overhead view of the main functional components of the bending assembly and their relationship to the rod being bent.

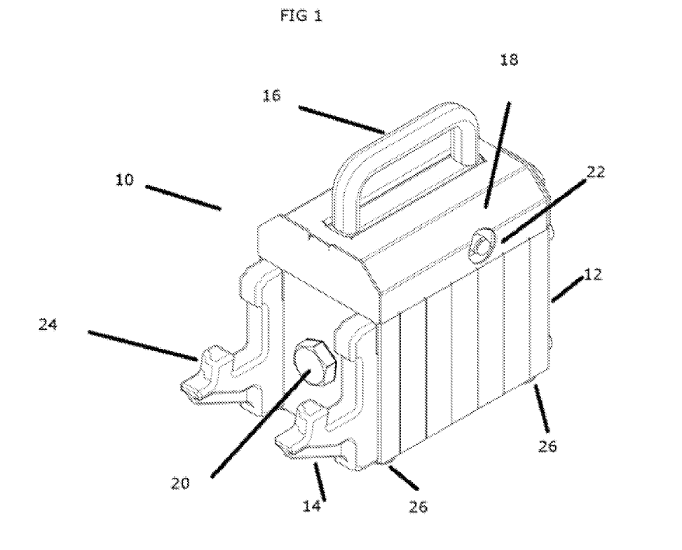

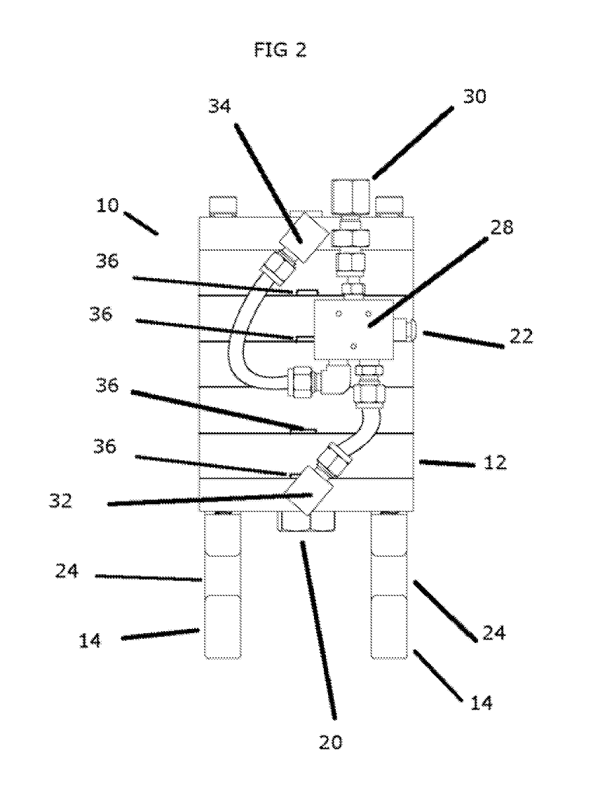

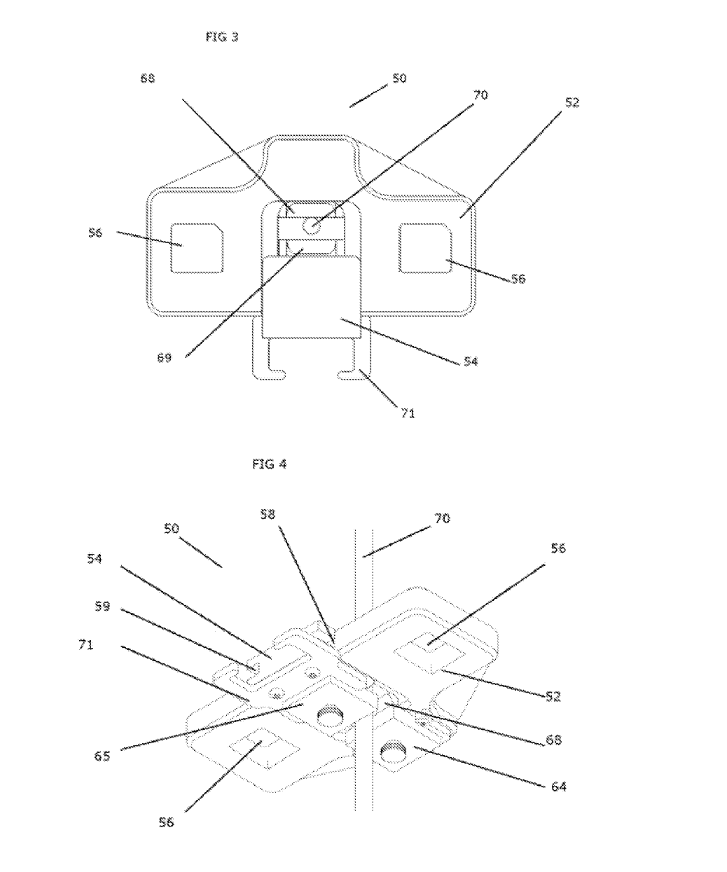

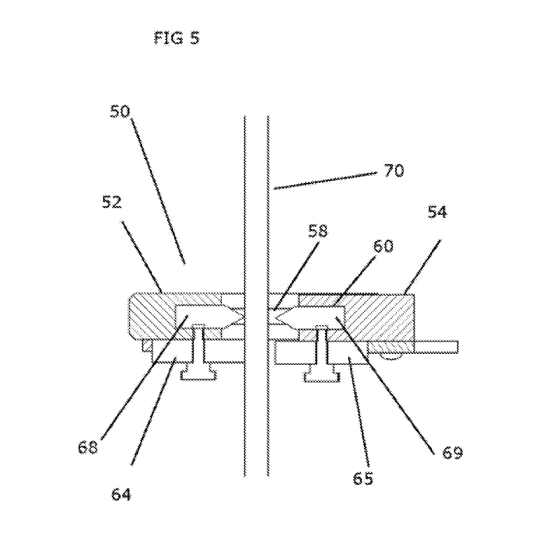

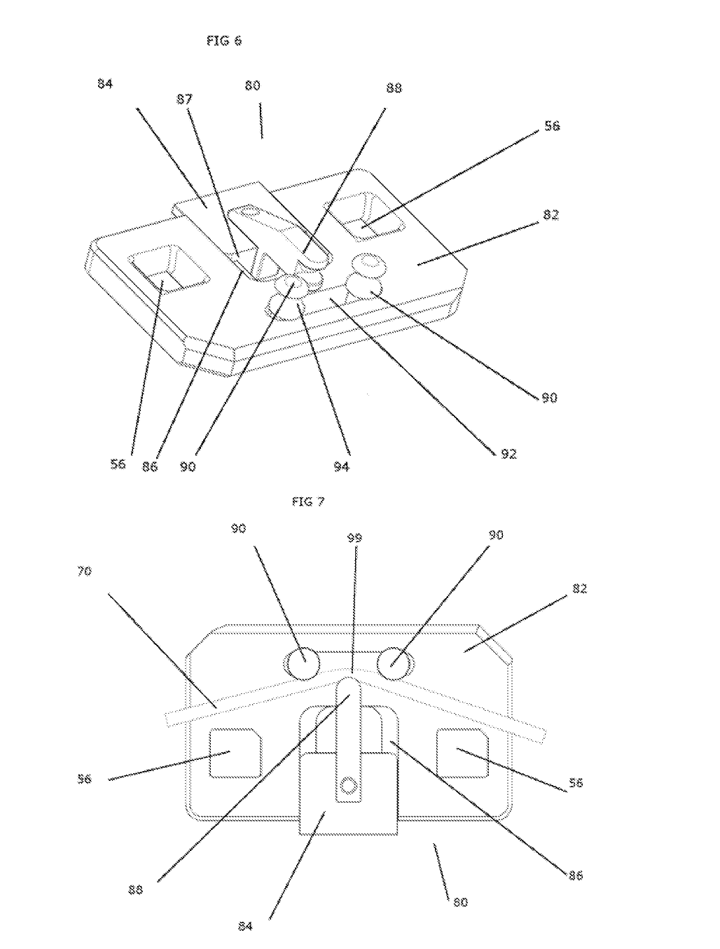

### DETAILED DESCRIPTION

### I. Overview

The automated rod manipulator consists of a power-base 10 as shown in the drawings that is a portable table top tool that is specifically designed and adapted for the manipulation of high strength surgical implant rods used for orthopedic surgery. The rod must be custom fitted to the patient during operation and consequently is required to be cut and bent quickly, cleanly, sterilely, and easily during the operation. Due to the strength of the metals used for surgical implants, large manual shear and wedge cutters and manual rod benders have been traditionally used. These have the disadvantages of being large and unwieldy, imprecise, and requiring significant muscular force and/or body weight to use. The automated rod manipulator consists of a power-base 10 and an array of attachments for manipulating the rod, including the cutting assembly 50 and bending assembly 80.

### II. Power-Base

The power-base 10 is the main power source for manipulating the rod. It is portable and easily moved around the operating room via a handle 16 and can be placed in a storage location or on a table on or off the sterile field resting on the stabilization feet 26. The power-base 10 is not sterilized in standard use, but is kept clean due to the protection provided by the cover 18. The main active components of the power-base include a pneumatic cylinder 12 attached to support platforms 14 which are meant to keep various attachments from moving away from the pneumatic cylinder 12 via retention hooks 24. The pneumatic cylinder 12 has a thrust piston 20 which interacts with cutting assembly 50 and bending assembly 80. The retention hooks 24 are angled towards the pneumatic cylinder 12 at approximately 10 degrees such that the cutting assembly 50 or bending assembly 80 are retained when force is applied in the direction of the thrust piston 20. An activation button 22 is used by the operator of the tool to release air pressure into the pneumatic cylinder 12 and move the thrust piston 20.

Plumbing underneath the cover 18 attaches an external compressed air source to pneumatic cylinder 12 via an actuation valve 28 which is controlled by the actuation button 22. The external compressed air source is attached to the power-base 10 via standard pneumatic adapters 30 found in operating rooms. With the activation button 22 undepressed, compressed air will flow into the pneumatic cylinder 12 via the retract port 32 and keep the thrust piston 20 securely stationary. Upon depressing the activation button 22, the compressed air will begin flowing through the activation valve 28 towards an extend port 34 and the compressed air will discontinue flowing to the retract port 32. The pressure differential will push the thrust piston 20 towards the retention hooks 24 located on the support platforms 14. When the thrust piston 20 moves, displaced air in the pneumatic cylinder 12 is exhausted through a series of exhaust ports 36 which deflect the exhaust away from a rod being manipulated. This simple motion of pressing the actuation button 22, which can be done with a single finger applying less than 3 lbs of force, causes the thrust piston 20 to impart force on the attachments to manipulate the high strength surgical rods.

The pneumatic cylinder used is a multi-stage varietal, such that the effective force required to cut or bend a rod can be achieved with compressed air found in an operating room. The current embodiment has approximately 5,000 lbs of force produced with 80 psi via a 5-stage pneumatic cylinder with a four inch piston diameter for each stage.

### III. Cutting Assembly

The rod cutting assembly 50 is comprised of several components including an external blade holder 52 which holds a static blade insert 68 stationary relative to the power-base 10 by locking the retention hooks 24 of the power-base 10 into the retention hook cavities 56. An internal blade holder 54 holds a dynamic blade insert 69 in a blade cavity 60. The static blade insert 68 and dynamic blade insert 69 have a 60 degree blade angle, allowing for the force produced by the power base 10 to cut a 6.5 mm diameter high strength surgical rod such as Ti-6Al-4V. The internal blade holder 54 slides into the external blade holder 52 using a corresponding set of male and female rail guides 58 and 59 respectively, which only allows for motion of the internal blade holder 54 and corresponding dynamic blade insert 69 in the direction of the thrust piston 20 once the blade assembly 50 is attached to the power-base 10. Additionally, an elastic internal eject-grip 65 is attached to the internal blade holder 54 and an external eject-grip 64 is attached to the external blade holder 52. A pullback mechanism 71 is part of the internal blade holder 54 and allows the internal blade holder 54 to follow the motion of the thrust piston 20 as it move back towards the pneumatic cylinder 12 after a cut.

### IV. Bending Assembly

The rod bending assembly 80 is comprised of several components including an external bender stage 82 which holds static pivots 90. An internal blade stage 84 holds the anvil 88 that moves in the same direction of the thrust piston 20. The internal bender stage 84 slides into the external bender stage 82 using a corresponding set of male and female rail guides 86 and 87 respectively, which only allows for motion of the internal bender stage 84 and corresponding anvil 88 in the direction of the thrust piston 20 once the bending assembly 80 is attached to the power-base 10. Although the static pivots 90 are held without moving in the direction of the thrust piston 20, the static pivots 90 can move laterally in the adjustment slots 92 to allow for adjustments in the resulting radius of curvature of the rod. Both the anvil 88 and static pivots 90 have rod aligning grooves 94 to allow the rod to seat securely during actuation.

### V. Cutting Operation

The power-base 10 must first be prepared by attaching the in house compressed air to the power-base 10 via the pneumatic adapters 30. For cutting a rod, the cutting assembly 50 must be sterilized then loaded onto the support platform 14. A rod 70 is then placed between the blades such that the desired length of rod is above the tip of the blades. The operator of the device pushes the actuation button 22 to move the thrust piston 20 that pushes against the internal blade holder 54 and moves the dynamic blade insert 69 and the internal eject-grip 65 towards the external blade holder 52, the static blade insert 68 and the external eject-grip 64. The rod 70 becomes pinched between the wedge shaped blade inserts 68 and 69 and cuts the rod 70 due to the force translated from the pneumatic cylinder 12. The internal eject-grip 65 moves with the dynamic blade insert 69 and elastically deforms around the rod 70 to retain the cut segment of the rod after the cut has been performed.

### VI. Bending Operation

The power-base 10 must first be prepared by attaching the in house compressed air to the pneumatic adapters 30. The rod bending assembly attachment 80 is first sterilized then loaded onto the support platform 14. The static pivots 90 are adjusted to a width that will produce the desired bend radius of curvature of the rod by moving them laterally in the adjustment slots 92. A rod 70 is then placed against the two static pivots 90 with the rod 70 securely seated in the rod aligning grooves 94. The operator of the device pushes the actuation button 22 to move the thrust piston 20 that pushes against the internal bender stage 84 and moves the anvil 88 towards the external bender stage 82 and the static pivots 90. The force translated from the pneumatic cylinder 12 deforms the rod 70 around the three pivot points made up of the static pivot points 90 and the anvil 88 causing a bend as shown on the rod at 99.

### VII. Alternative Embodiments

The above is limited to specific versions of the rod cutter and bender assembly and the power-base which affects the use of said assemblies. Alternative versions are possible. There is no requirement that all versions of the invention include the above-described features.

The pneumatic cylinder used in the above embodiment is a multi-stage varietal, such that the effective force required to cut or bend a rod can be achieved with compressed air found in an operating room. With a 60 degree blade, the force required to cut a 6.5 mm diameter Ti-6Al-4V rod is approximately 5,000 lbs of force, and therefore with 80 psi available in an operating room, the cylinder would have to have a diameter of 9″, or be a multi-stage equivalent. Different blade angles and cylinder sizes would allow for cutting of smaller rods with a lighter tool, or heavier or stronger rods with a more substantial tool.

While the above embodiment of the power-base cylinder is powered by pneumatic forces, another version may include electric or hydraulic power. These alternative power sources may be available in the operating room.

In some embodiments of the invention the geometric features of the blades that facilitate cutting of the rod may themselves differ from what is described above or differ in the relationship of said blades to each other in the static and dynamic positions. The geometric features of the cutting assembly may differ from what has been described as well to accommodate different sized blades or to retain those blades by different methods including the addition of materials such as set screws or other retaining devices.

The geometric features of the bending assembly specified in the present embodiment that facilitate changes in linearity of the rod may also differ in alternate embodiments. Static pivots may be made dynamic and vice versa. Static pivots may be made mobile by some other method including but not limited to lateral thread-adjusted positioning or self-locking mechanisms.

The association of the thrust piston to the internal blade holder is not limited to the embodiment of the invention herein described. Another embodiment may connect the thrust piston in a more permanent fashion to the internal blade holder or the blade itself.

In addition, the power-base and support platform are able to support interchangeable assemblies different from the above-described cutting and bending assemblies. Such assemblies may address surgical or non-surgical material manipulations, including but not limited to press molding of bone for the purposes of customized autograft bone creation.

Furthermore, the geometric features of the support platform are not limited to the current embodiment. The cutting or bending assemblies may be connected to the power-base in another fashion including permanent fixation to the power-base or by alternative means of fixation to the current support platform by some other means of retention which differs from the presently described retention hooks.

We claim:

1. An apparatus for the manipulation of surgical rods by cutting of a plurality of diameters comprising
a power-base comprising
a series of stabilization feet
a handle for portability of the apparatus
a connection for a compressed air supply
a series of plumbing (retraction-plumbing and extension-plumbing) connected to
a manually operated actuation valve including an actuation button and internal plumbing that allows the compressed air to fill
a pneumatic cylinder for the transfer of force to
a thrust piston transferring force to
a rod cutting blade assembly comprising
an internal blade assembly including
an internal eject-grip and an internal blade assembly movably connected to
an external blade assembly including
an external eject-grip and an external blade assembly removably attached to
a support frame firmly attached to the power-base.

2. The apparatus of claim 1 wherein said eject-grips contain the distal end of the rod to be cut preventing uncontrolled release of the distal rod segment during and immediately after cutting the rod.

3. The apparatus of claim 1 wherein said rod cutting assembly performs any number of repeated wedge cuts of a surgical rod without requiring the assistance of a second operator due to the automated pneumatic power-base when said rod is placed by the sole operator between the cutting blades during depression of the actuation button.

4. The apparatus of claim 1 wherein the blade assembly is removed from the power-base and sterilized separately from the power-base. The operator performs deconstruction of the cutting assembly including separation of the internal blade assembly from the external blade assembly and removal of the blades and eject-grips from both internal and external blade holders.

5. The apparatus of claim 1 wherein said blades do not require any maintenance or sharpening means said blades are replaced after each operation. The blades only require sterilization followed by installation prior to use for a single patient procedure.

6. The apparatus of claim 1 wherein said operation can be accomplished by a single operator whilst allowing the single operator line of sight of the cutting operation and the cutting blades to insure cutting at the point of interest.

7. An apparatus for the manipulation of surgical rods by bending a plurality of diameters comprising
a power-base comprising
a series of stabilization feet
a handle for portability of the apparatus
a connection for a compressed air supply
a series of plumbing (retraction-plumbing and extension-plumbing) connected to
a manually operated actuation valve including an actuation button and internal plumbing that allows the compressed air to fill
a pneumatic cylinder for the transfer of force to
a thrust piston transferring force to
a rod bending assembly comprising
an internal dynamic anvil movably connected to
an external bender stage including two pivots positionable in a plurality of adjustment slots removably attached to
a support frame firmly attached to the power-base.

8. The apparatus of claim 7 wherein the rod bending assembly is removed from the power-base and sterilized separately from the power-base. The operator performs deconstruction of the bending assembly including separation of the internal bender stage from the external bender stage and removal of the static pivot points.

9. The apparatus of claim 7 wherein said operation can be accomplished by any number of repeated rod bending operations without requiring the assistance of a second operator due to the automated, pneumatic power-base.

10. The apparatus of claims 1 and 7 wherein said power-base is covered with a sterile shroud while the sterilized cutting or bending assembly is placed on the shrouded support platform. A non-sterile operator can thus access the actuation button from beneath the sterile shroud while a second, sterile, operator can remain in the sterile field if such a case were to occur.

11. The apparatus of claims 1 and 7 wherein said handle provides portability of the apparatus and the stabilization feet provide non-marking, non skidding, dampening support to the apparatus.

12. The apparatus of claims 1 and 7 wherein said retention hooks are angled such that they mechanically retain the cutting assembly or bending assembly simply and without permanent fixation when when under load, but allowing removal of the assemblies when not under force from the piston.
